(19) United States
(12) Patent Application Publication
(10) Pub. No.: US 2004/0138428 A1
(43) Pub. Date: Jul. 15, 2004

(54) System and method for cleaving antibodies

(76) Inventor: Gerardo Zapata, San Mateo, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/688,198

(22) Filed: Oct. 17, 2003

Related U.S. Application Data
(60) Provisional application No. 60/419,908, filed on Oct. 18, 2002.

(51) Int. Cl. C12P 21/04; A61K 39/395; C07K 16/40; C12P 21/06

(52) U.S. Cl. 530/388.26; 435/70.21; 435/68.1

(57) ABSTRACT

The present invention is related to a method for producing antibody fragments. In particular, the invention involves a method for the production of F(ab′)₂ fragments. The method comprises concentration of cell culture media and activation of endogenous enzymes present in the cell culture media by adjusting the temperature and pH.

### RELATED APPLICATIONS

This application claims priority to U.S. Provisional Application No. 60/419,908 filed on Oct. 18, 2002, the disclosure of which is incorporated by reference herein it its entirety.

### BACKGROUND OF THE INVENTION

Summary of the Invention

The present invention is related generally to methods for generating antibody fragments. In particular, the invention relates to cleaving antibody molecules using endogenous enzymes present in cell culture medium.

Background of the Technology

The production of antibody fragments typically relies on the digestion of intact immunoglobulin molecules with particular enzymes. The type of antibody fragments that result from digestion of these immunoglobulins depends on the particular enzyme used in the digestion. For example, the production of two identical Fab′ antibody fragments, and a crystalline fragment (Fc), results from digestion of an antibody at a position above the disulfide linkage in the hinge region. The Fab′ antibody fragment includes a light chain and a portion of one of the heavy chains in the immunoglobulin and includes the specific antigen-binding sites. Enzymes, such as the cysteine proteinase papain, are useful for cleaving these type of disulfide linkages.

Other types of antibody fragments can be produced by cleaving the immunoglobulin at a position below the hinge region. For example, a single divalent F(ab′)₂ antibody fragment that has two antigen binding sites and a smaller Fc fragment will result from digesting an antibody below the hinge region. The Fc fragment includes the remaining portion of the heavy chains that is not responsible for antigen binding and is not included in the Fab or F(ab′)₂ antibody fragments. Enzymes such as the aspartyl proteinase pepsin will perform such a digestion.

Antibody fragments are typically used in immunoassays, immunotherapeutics and immunodiagnostics. Although antibody fragments provide advantages over whole antibodies, in order to be useful they should maintain the molecular integrity and binding properties of intact antibody.

In general, antibody fragments are prepared by incubating immunoglobulins with particular enzymes that digest the immunoglobulins into fragments. However, this method requires several incubation steps and the addition of expensive purified enzymes. Thus, what is needed in the art is an inexpensive and convenient way to generate antibody fragments.

### SUMMARY OF THE INVENTION

One aspect of the invention includes a method for generating F(ab′)₂ fragments. In particular, some advantageous embodiments involve expression of an immunoglobulin, such as IgG in cell culture, isolation of the cell culture media containing the IgG antibody, concentration of the cell culture media by ultra filtration through a filter, and initiation of cleavage of the IgG antibodies by activating enzymes in the cell culture media by adjusting the temperature and/or pH of the cell culture media. In some embodiments, the cell culture medium is concentrated 10 fold, the temperature is adjusted to about 37° C., and the pH is adjusted to about 3.5.

Another aspect of the invention includes a method of generating of F(ab′)₂ fragments of an antibody by inhibiting cysteine protease activity in the cell culture. In some embodiments, the cysteine protease activity is inhibited prior to initiating cleavage of the antibody molecule. In another aspect of the invention, the F(ab′)₂ fragments of an antibody are purified using anion and hydrophobic interaction chromatography.

Another aspect of the invention includes antibody fragments produced by a method containing the steps of: providing an antibody-producing cell line that is growing in a cell media under conditions to express antibodies; adjusting the conditions of the cell media to activate at least one enzyme that cleaves the antibodies; and incubating the cell line under the conditions so that the antibodies are cleaved into antibody fragments. In some advantageous embodiments, adjusting the conditions of the cell media includes adjusting the temperature or the pH of the cell media.

### BRIEF DESCRIPTION OF THE DRAWINGS

FIG. 1 is a line graph that shows a higher level of enzymatic activity measured using the Enzcheck protease activity kit (E-6638 from Molecular Probes) with a fermentation batch which contains a protein free media with peptone sources (circles) than with a fermentation batch which contains commercial media fortified with peptone sources (triangles).

FIG. 2 is a line graph that illustrates a chromatogram from size exclusion chromatography of the F(ab′)₂ and F(ab′)₂* mixtures produced by digestion of antibodies by proteinases activated at 37° C. and a pH of 3.5 in the cell culture medium of a cell culture expressing IgG. The chromatogram shows no separation or shoulders between the F(ab′)₂* smaller molecular weight fragment and the F(ab′)₂ fragment.

### DETAILED DESCRIPTION

Embodiments of the invention relate to methods for producing antibody fragments from intact immunoglobulin molecules. In particular, one embodiment involves the robust production of antibody fragments, including F(ab′)₂ fragments, from intact antibody molecules. In this embodiment, the antibody fragments are produced by activating endogenous enzymes in the cell culture medium that are secreting the antibodies. Subsequent purification of the antibody fragments results in purified products that may be used in in vitro therapeutic and diagnostic studies.

In one embodiment, enzymatic digestion of the secreted antibodies by aspartyl proteases, cysteinyl proteases, or a combination of both types of proteases is initiated by lowering the pH of the cell media to about pH 3.5 and adjusting the temperature to about 37° C. Once the antibodies have been digested by the activated enzymes, further digestion by the enzymes can be inhibited by altering the growth conditions. The particular endogenous enzyme that is activated in the media can be selected by varying the culture conditions. For cysteinyl proteases can be specifically and irreversibly inhibited adding cysteine protease inhibitors such as E-64 (Molecular Probes), or by increasing the pH of the media to 8.5 and incubating the reaction mixture for approximately two hours. Following this inactivation at pH 8.5, the media can be brought to pH 3.5 and 37° C. in order to specifically activate any aspartyl proteases in the media. Thus, this embodiment is useful for generating of F(ab′)₂ fragments since only the aspartyl proteases will act upon the immunoglobulins in the cell media.

A. Definitions

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used for recombinant DNA, oligonucleotide synthesis, and tissue culture and transformation (e.g., electroporation, lipofection).

Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)), which is incorporated herein by reference. The nomenclatures utilized in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

As utilized in accordance with the present disclosure, the following terms, unless otherwise indicated, shall be understood to have the following meanings:

“Antibody” or “immunoglobulin” or “antibody fragment” or “immunoglobulin fragment” refers to an intact antibody, or a binding fragment thereof that competes with the intact antibody for specific binding. Binding fragments of an antibody include Fab, Fab′, F(ab′)₂, Fv, and single-chain antibodies. An antibody other than a “bispecific” or “bifunctional” antibody is understood to have each of its binding sites identical. An antibody substantially inhibits adhesion of a receptor to a counterreceptor when an excess of antibody reduces the quantity of receptor bound to counterreceptor by at least about 20%, 40%, 60% or 80%, and more usually greater than about 85% (as measured in an in vitro competitive binding assay).

As discussed above, enzymatic digestion of antibodies by activation of an endogenous enzyme such as papain, or a similar enzyme, results in two identical antigen-binding fragments, known also as “Fab” fragments, and a “Fc” fragment, having no antigen-binding activity but having the ability to crystallize. Digestion of antibodies with the endogenous enzyme pepsin, or similar enzymes, results in a “F(ab′)₂” fragment in which the two arms of the antibody molecule remain linked and comprise two-antigen binding sites. The F(ab′)₂ fragment has the ability to crosslink antigen and has equivalent binding affinity to intact antibody molecules. Of course, embodiments of the invention are not limited to activation of any particular enzyme. Activation of any endogenous enzyme that cleaves an antibody is within the scope of the present invention.

“Fv” when used herein refers to the minimum fragment of an antibody that retains both antigen-recognition and antigen-binding sites. The region consists of a dimer of one heavy- and one light-chain variable domain in tight, non-covalent association. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

“Fab” when used herein refers to a fragment of an antibody which comprises the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. F(ab′)₂ antibody fragments originally were produced as pairs of Fab′ fragments which have hinge cysteines between them. Other chemical couplings of antibody fragments are also known.

“Pepsin-like aspartyl protease activity” or “aspartyl protease activity” when used herein refers to digestion of immunoglobulin molecules into F(ab′)₂ fragments. Specifically, the “aspartyl protease” or “aspartyl endopeptidase” digests the Fc portion of IgG₂ molecules and leaves defined F(ab′)₂ hinge terminals.

“Cysteinyl activity” or “cysteine enzyme activity” when used herein refers to digestion of the heavy chain of IgG₂ or additional digestion F(ab′)₂ molecules by a “cysteine enzyme” or “cysteine endopeptidase” or “cysteine proteinase” between the heavy chain variable domain and the constant 1 region of the heavy chain. The heavy chain cut in the F(ab′)₂ molecules that results from digestion by the cysteine enzyme digestion does not decrease the binding activity of the F(ab′)₂ molecule because the variable heavy domain remains attached to the light chain via a strong hydrophobic interaction.

“Antigen” when used herein refers to sequences that are responsible for specific binding of an antibody molecule to a particular target.

B. Antibody Structure

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., fourth ed. Raven Press, N.Y. (1998)) (incorporated by reference in its entirety for all purposes). The variable regions of each light/heavy chain pair form the antibody binding site.

Thus, an intact antibody has two binding sites. Except in bifunctional or bispecific antibodies, the two binding sites are the same.

The chains all exhibit the same general structure of relatively conserved framework regions (FR) joined by three hyper variable regions, also called complementarity determining regions or CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4. The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987); Chothia et al. Nature 342:878-883 (1989).

A bispecific or bifunctional antibody is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann Clin. Exp. Immunol. 79: 315-321 (1990), Kostelny et al. J. Immunol. 148:1547-1553 (1992). Production of bispecific antibodies can be a relatively labor intensive process compared with production of conventional antibodies and yields and degree of purity are generally lower for bispecific antibodies. Bispecific antibodies do not exist in the form of fragments having a single binding site (e.g., Fab, Fab′, and Fv).

C. Human Antibodies and Humanization of Antibodies

Human antibodies avoid certain of the problems associated with antibodies that possess murine or rat variable and/or constant regions. The presence of such murine or rat derived proteins can lead to the rapid clearance of the antibodies or can lead to the generation of an immune response against the antibody by a patient. In order to avoid the utilization of murine or rat derived antibodies, fully human antibodies can be generated through the introduction of human antibody function into a rodent so that the rodent produces fully human antibodies.

Human Antibodies

One method for generating fully human antibodies is through the use of XenoMouse™ strains of mice which have been engineered to contain 245 kb and 190 kb-sized germline configuration fragments of the human heavy chain locus and kappa light chain locus. See Green et al. Nature Genetics 7:13-21 (1994). The XenoMouse strains are available from Abgenix, Inc. (Fremont, Calif.).

The production of the XenoMouse is further discussed and delineated in U.S. patent application Ser. No. 07/466,008, filed Jan. 12, 1990; Ser. No. 07/610,515, filed Nov. 8, 1990; Ser. No. 07/919,297, filed Jul. 24, 1992; Ser. No. 07/922,649, filed Jul. 30, 1992; Ser. No. 08/031,801, filed Mar. 15, 1993; Ser. No. 08/112,848, filed Aug. 27, 1993; Ser. No. 08/234,145, filed Apr. 28, 1994; Ser. No. 08/376,279, filed Jan. 20, 1995; Ser. No. 08/430, 938, Apr. 27, 1995; Ser. No. 08/464,584, filed Jun. 5, 1995; Ser. No. 08/464,582, filed Jun. 5, 1995; Ser. No. 08/463,191, filed Jun. 5, 1995; Ser. No. 08/462,837, filed Jun. 5, 1995; Ser. No. 08/486,853, filed Jun. 5, 1995; Ser. No. 08/486,857, filed Jun. 5, 1995; Ser. No. 08/486,859; filed Jun. 5, 1995; Ser. No. 08/462,513, filed Jun. 5, 1995; Ser. No. 08/724,752, filed Oct. 2, 1996; and Ser. No. 08/759,620, filed Dec. 3, 1996 and U.S. Pat. Nos. 6,162,963, 6,150,584, 6,114,598, 6,075,181, and 5,939,598 and Japanese Patent Nos. 3 068 180 B2, 3 068 506 B2, and 3 068 507 B2. See also Mendez et al. Nature Genetics 15:146-156 (1997) and Green and Jakobovits J. Exp. Med. 188:483-495 (1998). See also European Patent No., EP 0 463 151 B1, grant published Jun. 12, 1996, International Patent Application No., WO 94/02602, published Feb. 3, 1994, International Patent Application No., WO 96/34096, published Oct. 31, 1996, WO 98/24893, published Jun. 11, 1998, WO 00/76310, published Dec. 21, 2000. The disclosures of each of the above-cited patents, applications, and references are hereby incorporated by reference in their entirety.

In an alternative approach, others, including GenPharm International, Inc., have utilized a “minilocus” approach. In the minilocus approach, an exogenous immunoglobin (Ig) locus is mimicked through the inclusion of pieces (individual genes) from the Ig locus. Thus, one or more V_{H} genes, one or more D_{H} genes, one or more J_{H} genes, a mu constant region, and a second constant region (preferably a gamma constant region) are formed into a construct for insertion into an animal. This approach is described in U.S. Pat. No. 5,545,807 to Surani et al. and U.S. Pat. Nos. 5,545,806, 5,625,825, 5,625,126, 5,633,425, 5,661,016, 5,770,429, 5,789,650, 5,814,318, 5,877,397, 5,874,299, and 6,255,458 each to Lonberg and Kay, U.S. Pat. Nos. 5,591,669 and 6,023,010 to Krimpenfort and Berns, U.S. Pat. Nos. 5,612,205, 5,721,367, and 5,789,215 to Berns et al., and U.S. Pat. No. 5,643,763 to Choi and Dunn, and GenPharm International U.S. patent application Ser. No. 07/574,748, filed Aug. 29, 1990, Ser. No. 07/575,962, filed Aug. 31, 1990, Ser. No. 07/810,279, filed Dec. 17, 1991, Ser. No. 07/853,408, filed Mar. 18, 1992, Ser. No. 07/904,068, filed Jun. 23, 1992, Ser. No. 07/990,860, filed Dec. 16, 1992, Ser. No. 08/053,131, filed Apr. 26, 1993, Ser. No. 08/096,762, filed Jul. 22, 1993, Ser. No. 08/155,301, filed Nov. 18, 1993, Ser. No. 08/161,739, filed Dec. 3, 1993, Ser. No. 08/165,699, filed Dec. 10, 1993, Ser. No. 08/209,741, filed Mar. 9, 1994, the disclosures of which are hereby incorporated by reference. See also European Patent No. 0 546 073 B1, International Patent Application Nos. WO 92/03918, WO 92/22645, WO 92/22647, WO 92/22670, WO 93/12227, WO 94/00569, WO 94/25585, WO 96/14436, WO 97/13852, and WO 98/24884 and U.S. Pat. No. 5,981,175, the disclosures of which are hereby incorporated by reference in their entirety. See further Taylor et al., 1992, Chen et al., 1993, Tuaillon et al., 1993, Choi et al., 1993, Lonberg et al., (1994), Taylor et al., (1994), and Tuaillon et al., (1995), Fishwild et al., (1996), the disclosures of which are hereby incorporated by reference in their entirety.

Kirin has also demonstrated the generation of human antibodies from mice in which, through microcell fusion, large pieces of chromosomes, or entire chromosomes, have been introduced. See European Patent Application Nos. 773 288 and 843 961, the disclosures of which are hereby incorporated by reference.

Human anti-mouse antibody (HAMA) responses have led the industry to prepare chimeric or otherwise humanized antibodies. While chimeric antibodies have a human constant region and a murine variable region, it is expected that certain human anti-chimeric antibody (HACA) responses will be observed, particularly in chronic or multi-dose utilizations of the antibody. Thus, it would be desirable to provide fully human antibodies against an antigen of interest in order to vitiate concerns and/or effects of HAMA or HACA response.

D. Design and Generation of Other Therapeutics

The antibody fragments that are produced by the methods described herein are particularly useful for coupling various labels thereto, such as radiolabels and fluorescent labels, according to methods known in the art for use as labeled reagents in immunoassays. Further uses for the antibody fragments include in vivo use as immunotherapeutics, such as immunotoxins, as peptide therapeutics and as antisense therapeutics and as in vivo immmunodiagnostics.

In connection with the generation of advanced antibody therapeutics, where complement fixation is a desirable attribute, it may be possible to sidestep the dependence on complement for cell killing through the use of bispecifics, immunotoxins, or radiolabels, for example.

In connection with immunotoxins, antibody fragments can be modified to act as immunotoxins utilizing techniques that are well known in the art. See e.g., Vitetta Immunol Today 14:252 (1993). See also U.S. Pat. No. 5,194,594. In connection with the preparation of radiolabeled antibody fragments, such modified antibodies can also be readily prepared utilizing techniques that are well known in the art. See e.g., Junghans et al. in Cancer Chemotherapy and Biotherapy 655-686 (2d edition, Chafner and Longo, eds., Lippincott Raven (1996)). See also U.S. Pat. Nos. 4,681,581, 4,735,210, 5,101,827, 5,102,990 (RE 35,500), 5,648,471, and 5,697,902. Each of immunotoxins and radiolabeled molecules would be likely to kill cells expressing the antigen of interest, and particularly those cells in which the antibodies of the invention are effective.

E. Preparation of Antibodies

Antibodies in accordance with the invention were prepared through the utilization of the XenoMouse technology, as described below. Such mice, then, are capable of producing human immunoglobulin molecules and antibodies and are deficient in the production of murine immunoglobulin molecules and antibodies. Technologies utilized for achieving the same are disclosed in the patents, applications, and references disclosed in the Background, herein. In particular, however, a preferred embodiment of transgenic production of mice and antibodies therefrom is disclosed in U.S. patent application Ser. No. 08/759,620, filed Dec. 3, 1996 and International Patent Application Nos. WO 98/24893, published Jun. 11, 1998 and WO 00/76310, published Dec. 21, 2000, the disclosures of which are hereby incorporated by reference. See also Mendez et al. Nature Genetics 15:146-156 (1997), the disclosure of which is hereby incorporated by reference.

Through use of such technology, fully human monoclonal antibodies against a variety of antigens have been produced. Essentially, the XenoMouse™ lines of mice were immunized with an antigen of interest, lymphatic cells (such as B-cells) were recovered from the mice that expressed antibodies, the recovered cells were fused with a myeloid-type cell line to prepare immortal hybridoma cell lines, the such hybridoma cell lines were screened and selected to identify hybridoma cell lines that produced antibodies specific to the antigen of interest.

In general, antibodies produced by the above-mentioned cell lines possessed fully human IgG2 heavy chains with human kappa light chains. The antibodies possessed high affinities, typically possessing Kd's of from about 10⁻⁶ through about 10⁻¹¹ M, when measured by either solid phase and solution phase.

As will be appreciated, antibodies can be expressed in cell lines other than hybridoma cell lines. Sequences encoding particular antibodies can be used for transformation of a suitable mammalian host cell. Transformation can be by any known method for introducing polynucleotides into a host cell, including, for example packaging the polynucleotide in a virus (or into a viral vector) and transducing a host cell with the virus (or vector) or by transfection procedures known in the art, as exemplified by U.S. Pat. Nos. 4,399,216, 4,912,040, 4,740,461, and 4,959,455 (which patents are hereby incorporated herein by reference). The transformation procedure used depends upon the host to be transformed. Methods for introduction of heterologous polynucleotides into mammalian cells are well known in the art and include dextran-mediated transfection, calcium phosphate precipitation, polybrene mediated transfection, protoplast fusion, electroporation, encapsulation of the polynucleotide(s) in liposomes, and direct microinjection of the DNA into nuclei.

Mammalian cell lines available as hosts for expression and secretion of antibodies are well known in the art and include many immortalized cell lines available from the American Type Culture Collection (ATCC), including but not limited to Chinese hamster ovary (CHO) cells, HeLa cells, baby hamster kidney (BHK) cells, monkey kidney cells (COS), human hepatocellular carcinoma cells (e.g., Hep G2), and a number of other cell lines. Cell lines of particular preference are selected through determining which cell lines have high expression levels and produce antibodies with constitutive specific binding properties.

Antibodies in accordance with the present invention are capable of binding to a particular antigen of interest. Further, antibodies of the invention are useful in the detection of antibodies and antigens in patient samples and accordingly are useful as diagnostics as described hereinbelow.

F. Preparation of Antibody Fragments

According to the present invention, a robust method for producing antibody fragments involves expression of antibodies of interest in a cell culture. The cell culture may be harvested by removing particulate matter and cells using depth filtration. After clarification, the cell culture media may be concentrated approximately 10× and stored prior to digestion.

Enzymatic digestion by aspartyl and cysteinyl proteases in the clarified and concentrated cell culture media can be initiated by lowering the pH to about 3.5 and lowering the temperature to 37° C. Irreversible inhibition of any cysteine enzymatic digestion can be performed by contacting the culture media with E-64 (Molecular Probes, Eugene, Oreg.) or by increasing the pH to 8.5 and incubating the reaction mixture for about two hours to activate any desired aspartyl enzymatic digestion in order to generate F(ab′)₂ fragments. Irreversible inhibition of any aspartyl enzymatic digestion can performed by adding an aspartyl enzyme inhibitor, such as Pepstatin (Aldrich), to the reaction mixture prior to lowering the pH to 3.5 and the temperature to 37° C.

The resulting digested products can be further purified by a number of purification methods including filtration, protein A chromatography and hydrophobic interaction chromatography (HIC) and further processed for use in therapeutics and diagnostics.

### EXAMPLES

The following examples, including the experiments conducted and results achieved are provided for illustrative purposes only and are not to be construed as limiting upon the present invention.

### Example 1

### Method for Cleaving Immunoglobulins

A. Process

Cells clones expressing immunoglobulins were selected from hybridoma or CHO cell cultures for use in a method for cleaving immunoglobulins.

1. Immunoglobulin-Expressing Cell Culture

a. Hybridoma Cell Culture

A hybridoma cell line was created by fusion of B-cells from XenoMouse animals with the non-secretory myeloma, P3X63Ag8.653, cell line (ATCC, cat. # CRL 1580, Kearney et al, J. Immunol. 123, 1979, 1548-1550). After selection of the chosen hybridoma clone, the clone was adapted to serum-free growth conditions using CD-hybridoma (Gibco-Invitrogen) growth medium. For the production of the antibody, cells were grown in stirred tank bioreactors using CD-hybridoma medium supplemented with glucose, glutamine and proteose peptone No 3 (Becton Dikinson). Cell culture supernatant was harvested by filtration or centrifugation and passed trough a sterile filter prior to being subjected to the pH treatments and activation of enzymatic cleavage.

b. CHO Cell Culture

CHO-DG44 cells were received from Dr. Larry Chasin, Columbia University, 912 Fairchild Center for Life Sciences, 1212 Amsterdam Ave., New York, N.Y. 100027 (Urlaub, G et al., Cell, 33: 405-412, 1983 and Urlaub, G et al., Somatic Cell and Molec. Gent., 12: 555-566, 1986). Cells were adapted to serum-free growth conditions using CHO-S SFM II culture medium (Gibco-Invitrogen). Cells were transfected with vectors coding for the light and heavy chains of a fully human antibody using the lipofectamine procedure (Gibco-Invitrogen). Cell clones were selected for expression of antibody. For the production of the antibody, cells were grown in stirred tank bioreactors using CD-CHO medium (Gibco-Invitrogen) supplemented with glucose, glutamine, pluronic F68, IGF-1 and proteose peptone No 3 (Becton Dikinson). Cell culture supernatant was harvested by filtration or centrifugation and passed through a sterile filter to remove particulate matter and cells prior to being subjected to the pH treatments and activation of enzymatic cleavage.

After filtration or clarification of the cell culture fermentation broth, the cell culture media was concentrated approximately 10 fold and further stored at 4-8° C. prior to digestion.

2. Cleavage of Immunoglobulins

To initiate enzymatic digestion in the cell culture media, the temperature of the clarified and concentrated cell culture fluid was adjusted to 37° C. in a stainless steel tank with a water jacket. After the temperature was stable, the pH of the cell culture fluid was lowered to approximately pH 3.5 using 6N HCl. Small adjustments to the pH were made with 5N NaOH and 6N HCl. Aliquots were taken at different pH values, for example, pH 5.0, 4.5, 4.0, 3.5, 3.0 and 2.5 and further loaded onto precast 10% and 4-20% bis-tris polyacrylamide gradient gels and subjected to either reduced or non-reduced SDS-PAGE electrophoresis which separates polypeptides according to molecular size and visualization by colloidal blue staining. Prior to loading on the polyacrylamide gels, samples were denatured by treatment with SDS. For reduced SDS-PAGE electrophoresis, samples were further treated with antioxidant which disrupted any disulfide bonds prior to loading the sample on the polyacrylamide gels.

Visualization of the SDS-PAGE electrophoresis gels indicated that maximal enzymatic activity of endogenous enzymes in the cell culture medium of a cell culture expressing IgG immunoglobulin occurred at a pH of 3.5.

B. Characterization of Method for Cleaving Immunoglobulins

To determine the level of digestion and to quantify the level of enzymatic activity that occured during this method of cleaving immunoglobulins, clarified and concentrated cell culture media was adjusted to a temperature of about 37° C. and a pH of about 3.5. Aliquots were taken at specific intervals for a time period of 22 hours and adjusted to a pH of 7.0 to stop the digestion prior to subjection to further characterization.

1. Level of Digestion

To determine the level of digestion, aliquots from the clarified, concentrated and activated cell culture medium that were taken every 0.5 to 1.0 hours during the 22 hour activation were subjected to either HPLC assay or SDS PAGE analysis.

For HPLC analysis, which distinguished monomeric IgG from larger molecular weight aggregates, test samples were injected onto a TosoHaas, TSK-Gel G3000SWXL HPLC column equilibrated in 0.2M sodium phosphate mobile phase (pH 7.0). Protein peaks were monitored at 280 nm and further analyzed by the integration system.

For SDS PAGE analysis, samples were loaded onto a 10% polyacrylamide gel and subjected to SDS PAGE electrophoresis and visualization by colloidal blue staining. Control samples representing standard F(ab′)₂ were also loaded onto the polyacrylamide gel and served as a control for comparison with the test samples.

Visualization of the SDS-PAGE gel indicated that the digestion of IgG immunoglobulin in cell culture medium of a cell culture expressing IgG immunoglobulin was completed in approximately 5 hours upon activation of digestion at 37° C. and a pH of 3.5. Additional experiments indicated that the time for complete digestion into F(ab′)₂ fragments varied depending on a number of conditions including the fermentation conditions.

2. Enzymatic Activity

To determine the level of enzymatic activity in a clarified, concentrated and activated cell culture, aliquots from a clarified, concentrated and activated cell culture were measured for enzymatic activity of the serine and cysteine proteases using the EnzCheck protease assay kit (E 6638, Molecular Probes, Eugene, Oreg.) according to the manufacturer's instructions.

a. Fermentation Media

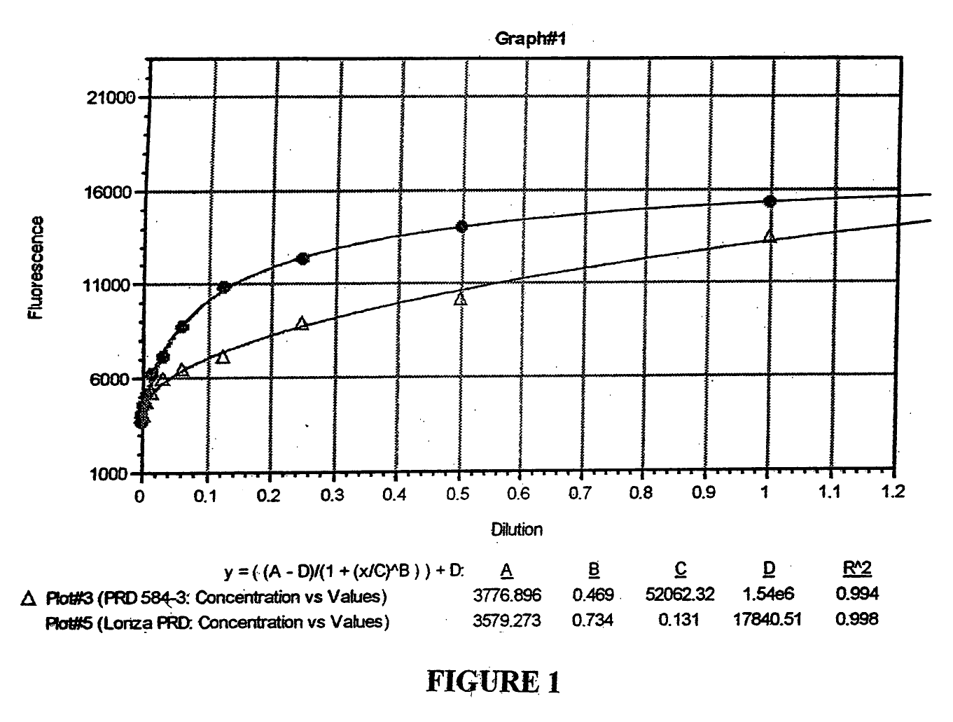

The level of enzymatic activity in clarified, concentrated and activated cell culture medium that involved different fermentation conditions was examined. Enzymatic activity in fermentation conditions involving commercial media fortified with peptone sources or protein free media without peptone sources was examined using the N-check protease assay method. Fermentation conditions including protein free media without peptone sources showed a higher enzymatic activity than fermentation conditions including commercial media that was fortified with peptone sources (FIG. 1).

b. Storage of Cell Culture Media

The level of enzymatic activity in clarified, concentrated and activated cell culture medium that involved storage of the cell culture medium for several weeks at 4-8° C. prior to activation was determined. Differences in enzymatic activity in clarified, concentrated and activated cell culture that involved different storage times of the cell culture medium prior to use was observed. Accordingly, determining the level of enzymatic activity of the enzymes in the cell culture media was helpful in determining the appropriate digestion time and temperature conditions for activation of the cell culture to generate F(ab′)₂ fragments.

c. Enzymes Responsible for Enzymatic Activity

The enzymes responsible for the enzymatic activity in the activated cell culture media were determined to be two different type of enzymes, an aspartyl and a cysteinyl protease.

To confirm the identity of the enzymes responsible for the enzymatic activity, the ability of specific protease inhibitors to affect cleavage of antibodies in the media was tested. The aspartyl enzyme inhibitor Pepstatin (Aldrich) and the cysteinyl protease inhibitor E-64 (Molecular Probes) were analyzed for their ability to inhibit, or reduce, protease activity in activated cell culture media. Isolation of aspartyl protease from the activated cell culture media was also performed using affinity pepstatin purification resin (Pierce).

Pepstatin or E64 was added to the digestion mixture prior to activation of the cell culture medium. The products produced after digestion in the presence of pepstatin or E64 were subjected to SDS-PAGE analysis and compared to a control pH 7.5 cell culture media sample and control samples isolated after digestion at a pH of 3.5 and a temperature of 37° C. for 4 hours in the absence of pepstatin.

Visualization of the SDS-PAGE gels showed inhibition of aspartyl enzymatic activity by pepstatin. Intact IgG2 molecules were observed in lanes that were loaded with aliquots taken from the cell culture medium after an incubation of 4 hours at a pH of 7.5. Intact IgG was also observed in lanes that were loaded with cell culture medium that was taken after an incubation of 4 hours at a pH of 3.5 and a temperature of 37° C. and in the presence of pepstatin. Lanes containing cell culture medium that was taken after an incubation in the presence of E64 showed a reduction in the formation of F(ab′)₂* fragments. Accordingly, the enzyme responsible for the production of F(ab′)₂* fragments was identified as the cysteinyl enzyme.

d. Control of Enzymatic Activity

As one desired product of the clarified, concentrated and activated cell culture medium is the 100 kD F(ab′)₂ product which is the result of the aspartyl enzyme activity described above, the cysteinyl enzyme activity which results in the formation of the F(ab′)₂* fragment can be prevented by several methods.

Inactivation of the cysteinyl enzyme in the cell culture medium was performed in one method using E64, the cysteinyl enzyme inhibitor. In a second method the cysteinyl enzyme in the cell culture media was initially activated by bringing the media to a low pH for a short period of time, followed by increasing the pH to 8.5 and incubating for two hours. After the media was incubated at pH 8.5, irreversible inactivation of the cysteinyl enzymatic activity was achieved. The pH of the cell culture media was then lowered to pH 3.5 to allow for the aspartyl enzyme activity which was not inhibited by the incubation at pH 8.5. F(ab′)₂ fragments, without F(ab′)₂* fragments, resulted from this incubation.

3. Products of Enzymatic Digestion

The products of the enzymatic digestion in the clarified, concentrated and activated cell culture media was determined by subjecting the products of a clarified, concentrated and activated cell culture medium produced after activation for 23 hours to SDS-PAGE analysis.

Essentially, the cell culture fermentation media from cells expressing IgG2 was harvested and concentrated. The temperature of the concentrated media was adjusted to 37° C. and the pH of the concentrated media was reduced to a pH of about 3.5 to initiate enzymatic digestion. The digestion reaction proceeded for 23 hours. Aliquots that were taken at specific time points and a molecular weight standard and a F(ab′)₂ fragment molecule standard were subjected to SDS-PAGE analysis.

Visualization of the SDS-PAGE gel indicated that the intact IgG2 molecule present at the beginning of the activation was digested during the activation and resulted in a F(ab′)₂ fragment of approximately 100 kDa that was generated after 8 hours of activation of the endogenous enzymes and a F(ab′)₂* fragment of approximately 75 kDa that was generated after 23 hours of activation of the endogenous enzymes.

Comparison of the products of the enzymatic digestion were performed in non-reducing and reducing conditions and analyzed by HPLC-MS and SDS-PAGE.

After reduction of the F(ab′)₂ fragments with DTT and separation analysis on HPLC-MS, the light chain and heavy chain from the F(ab′)₂ fragment were 23329 Da and 26440 Da, respectively. After reduction of the F(ab′)₂* fragments with DTT and separation analysis on HPLC-MS, the light chain from the F(ab′)₂* fragments was 23329 Da and the heavy chain from the F(ab′)₂* fragments was 14776 Da and 11664 Da.

An intact IgG2 control sample, the products of the enzymatic digestion of the cell culture media with cysteinyl activity inhibited, primarily F(ab′)₂ fragments, and the products of the enzymatic digestion of the cell culture media without inhibition of cysteinyl activity, both F(ab′)₂ and F(ab′)₂* fragments, were subjected to SDS-PAGE analysis under non-reducing and reducing conditions. The SDS-PAGE analysis shows that the heavy chain of the F(ab′)₂* fragment separating into a 14776 Da fragment and al 11664 fragment which run approximately at the 14 kD molecular weight marker.

### Example 2

### Purification of F(ab′)₂ Fragments

For purification of the F(ab′)₂ fragments generated in the digestion reaction, the cell culture media was further subjected to a number of chromatography steps including a Q Sepharose FF column (Amersham Pharmacia), a protein A column, and a hydrophobic interaction chromatography (HIC) column.

A. Q Sepharose FF Column

As host cell proteins in the cell culture media precipitated during the low pH activation of digestion, the protein precipitate was removed prior to protein chromatography using depth filtration with a Milliguard CWSC filter (Millipore). The cell culture fluid was then adjusted to pH 8.0±1 using 1M Tris pH 8.0, and the conductivity of the cell culture fluid was adjusted to approximately 10 mS. The cell culture fluid was loaded onto the Q Sepharose FF column (Amersham Pharmacia) which was equilibrated in 20 mM Tris, pH 8.0. After loading of the cell culture fluid, the column was washed with 5 column volumes (CV) of equilibrated buffer. The antibody fragment product was eluted with a 15 CV gradient from 20 mM Tris pH 8.0 to 20 m Tris, 500 mM NaCl, pH 8.0. The product was collected in fractions and the fractions were analyzed by SDS PAGE.

B. Protein A Column

To further remove any residual IgG2 remaining in the eluted product, the Q Sepharose pool was conditioned by adjusting the pH to 7.4±0.1 and loaded onto a Protein A column which was equilibrated with PBS pH 7.4. After loading the Q Sepharose FF pool onto the Protein A column, the Protein A column was washed with 5 CVs of PBS equilibration buffer. Aliquots containing the flow through were analyzed for the presence of the protein product by measuring the absorbance at A280 of the aliquots collected. The aliquot containing the product was referred to as the Protein A pool

C. Hydrophobic Interaction Chromatography (HIC) Column

The Protein A pool was conditioned by adding an equal volume of PBS/3M (NH₄)₄SO₄, pH 7.0 and adjusting the pH to 7.0±0.1 and then loaded onto a hydrophobic interaction chromatography (HIC) column that was equilibrated in PBS/11M (NH₄)₄SO₄, pH 7.0. After loading the Protein A pool onto the HIC column, the HIC column was washed with 5 CV of equilibration buffer, followed by elution of the product using a gradient from PBS/3M (NH₄)₄SO₄, pH 7.0 to PBS pH 7.0. The eluted product was collected in fractions which were further subjected to SDS PAGE analysis.

The aliquots taken during the purification process of the activated cell culture medium along with an intact IgG2 control were subjected to non-reduced SDS-PAGE electrophoresis or subjected to reduction with DTT and further to reduced SDS-PAGE electrophoresis. In the non-reduced gel, the purified approximately 100 kDa F(ab′)₂ and the purified approximately 75 kDa F(ab′)₂* fragments produced by digestion of the aspartyl enzymes and cysteine enzymes, respectively, were visualized on the non-reduced SDS-PAGE gels. In the reduced gel, the 23 kDa light chain and 26 kDa heavy chain from the F(ab′)₂ fragment and the 23 kDa light chain and the 14 kDa and 11 kDa fragments of the heavy chain of F(ab′)₂* which both run at the 14 kDa molecular weight marker were visualized.

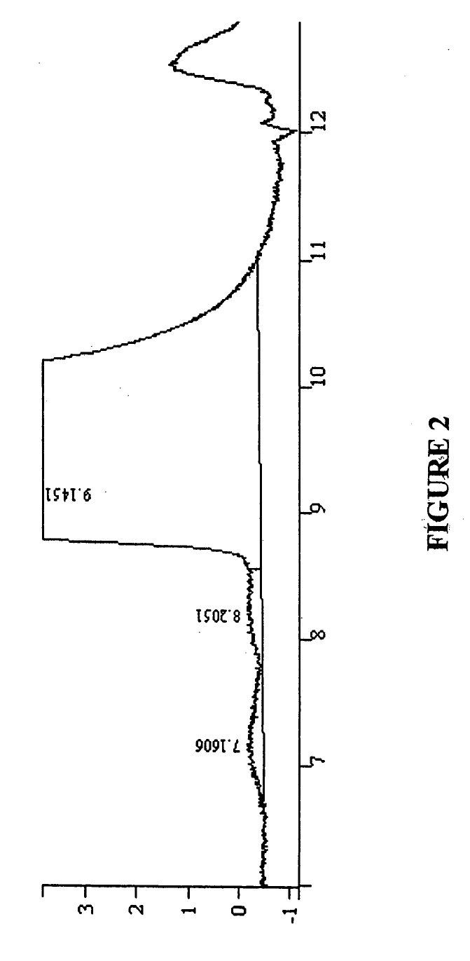

The size exclusion chromatogram of the cell culture medium shows no separation or shoulders between the F(ab′)₂* smaller molecular weight fragment and the F(ab′)₂ fragment suggesting that the 14 KD fragment clipped in the F(ab′)₂* molecule remains attached to the antibody fragment by strong hydrophobic interactions (FIG. 2).

The foregoing written specification is considered to be sufficient to enable one skilled in the art to practice the invention. The present invention is not to be limited in scope by the construct deposited, since the deposited embodiment is intended as a single illustration of certain aspects of the invention and any constructs that are functionally equivalent are within the scope of this invention. The deposit of material herein does not constitute an admission that the written description herein contained is inadequate to enable the practice of any aspect of the invention, including the best mode thereof, nor is it to be construed as limiting the scope of the claims to the specific illustrations that it represents.

### INCORPORATION BY REFERENCE

All references cited herein, including patents, patent applications, papers, test books, and the like, and the references cited therein, to the extent that they are not already, are hereby incorporated herein by reference in their entirety. In addition, the following references are also incorporated by reference herein in their entirety, including the references cited in such references.

### EQUIVALENTS

The foregoing description and Examples detail certain preferred embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

What is claimed is:

1. A method for generating fragments of an antibody, comprising:
providing an antibody-producing cell line that is growing in a cell media under conditions to express antibodies;
adjusting the conditions of the cell media to activate at least one endogenous enzyme that cleaves said antibodies; and
incubating said cell line under said conditions so that said antibodies are cleaved into antibody fragments.

2. The method of claim 1, wherein said antibodies are cleaved into F(ab′)₂ fragments.

3. The method of claim 1, wherein adjusting the conditions of the cell media comprises adjusting the temperature of the cell media.

4. The method of claim 1, wherein adjusting the conditions of the cell media comprises adjusting the pH of the cell media.

5. The method of claim 4, wherein adjusting the pH comprises adjusting the pH to about pH 3.5.

6. The method of claim 1, further comprising inactivating said at least one endogenous enzyme after incubating said cell line.

7. The method of claim 1, further comprising substantially purifying said antibody fragments by affinity chromatography.

8. The method of claim 1, wherein said at least one enzyme comprises a serine protease.

9. The method of claim 1, wherein said at least one enzyme comprises a cysteine protease.

10. The method of claim 1, wherein said at least one enzyme comprises an aspartyl protease.

11. The method of claim 1 wherein the cell line comprises cells selected from the group consisting of: Chinese hamster ovary cells, HeLa cells, baby hamster kidney cells, monkey kidney cells, and human hepatocellular carcinoma cells.

12. The method of claim 1 wherein the cell line comprises CHO-DG44 cells.

13. The method of claim 1 wherein the cell media is a protein free media.

14. The method of claim 1 wherein the cell media comprises a peptone source.

15. The method of claim 1 wherein the cell media is a CD-CHO media.

16. The method of claim 1 further comprising inactivating said at least one enzyme by adjusting pH.

17. The method of claim 16 wherein inactivating said at least one enzyme comprises inactivating a cysteinyl enzyme.

18. The method of claim 17 further comprising activating an aspartyl enzyme by adjusting the pH of the cell media after endogenous cysteinyl enzyme activity has been reduced.

19. A method for producing F(ab′)₂ fragments of an antibody, comprising:
providing a cell media comprising a cell line that is growing under conditions to produce a recombinant antibody;
inactivating endogenous cysteinyl enzyme activity in said cell media; and
activating endogenous aspartyl enzyme activity in said cell media, wherein said activation results in cleavage of said recombinant antibody into F(ab′)₂ fragments.

20. The method of claim 19 wherein the cell media is a CD-CHO media.

21. The method of claim 19, wherein inactivating endogenous cysteinyl enzyme activity comprises adjusting the pH of the cell media.

22. The method of claim 19, wherein inactivating endogenous cysteinyl enzyme activity comprises adding a cysteinyl enzyme inhibitor to the cell media.

23. The method of claim 22, wherein cysteinyl enzyme inhibitor is E64.

24. The method of claim 19, wherein activating endogenous aspartyl enzyme activity comprises adjusting the pH of the cell media.

25. The method of claim 19, further comprising purifying said F(ab′)₂ fragments from said cell media.

26. Antibody fragments produced by a method comprising the steps of:
providing an antibody-producing cell line that is growing in a cell media under conditions to express antibodies;
adjusting the conditions of the cell media to activate at least one enzyme that cleaves said antibodies; and
incubating said cell line under said conditions so that said antibodies are cleaved into antibody fragments.

27. The antibody fragments according to claim 26 wherein adjusting the conditions of the cell media comprises adjusting the temperature of the cell media.

28. The antibody fragments according to claim 26 wherein adjusting the conditions of the cell media comprises adjusting the pH of the cell media.

29. The antibody fragments according to claim 26 wherein adjusting the pH comprises adjusting the pH to about pH 3.5.
